(19) United States
(12) Patent Application Publication
(10) Pub. No.: US 2024/0260889 A1
(43) Pub. Date: Aug. 8, 2024

(54) MOISTURE MEASURING DEVICE

(71) Applicant: Murata Manufacturing Co., Ltd., Kyoto (JP)

(72) Inventors: Tomoki TAKAHASHI, Kyoto (JP); Jun TAKAGI, Kyoto (JP); Koji TANAKA, Kyoto (JP)

(21) Appl. No.: 18/616,985

(22) Filed: Mar. 26, 2024

Related U.S. Application Data
(63) Continuation of application No. PCT/JP2022/030986, filed on Aug. 16, 2022.

(30) Foreign Application Priority Data
Sep. 28, 2021 (JP) 2021-158191

(51) Int. Cl. A61B 5/00; A61B 5/0538

(52) U.S. Cl. CPC A61B 5/4277; A61B 5/0538; A61B 2562/0214

(57) ABSTRACT

A moisture measuring device includes a biosensor, a processing unit, a substrate, a housing, and one or more attaching members. The biosensor has a sensor surface for acquisition of biological information. The processing unit is configured to process the biological information acquired by the biosensor. The substrate has a first principal surface on which the processing unit is mounted. The housing accommodates the biosensor, the processing unit, and the substrate. The one or more attaching members are disposed on the first principal surface side of the substrate to attach the substrate to the housing. The total area of one or more overlap regions in which the one or more attaching members overlap the substrate in the first principal surface of the substrate when viewed in a direction perpendicular to the first principal surface of the substrate is smaller than a sensor area of the sensor surface of the biosensor.

### CROSS REFERENCE TO RELATED APPLICATION

This is a continuation of International Application No. PCT/JP2022/030986 filed on Aug. 16, 2022 which claims priority from Japanese Patent Application No. 2021-158191 filed on Sep. 28, 2021. The contents of these applications are incorporated herein by reference in their entireties.

### BACKGROUND OF THE DISCLOSURE

### Field of the Disclosure

The present disclosure relates to a moisture measuring device.

### Description of the Related Art

An intraoral moisture measuring instrument is disclosed in Patent Document 1. The intraoral moisture measuring instrument disclosed in Patent Document 1 includes a swing member, a moisture detection unit provided at a tip of the swing member, and a biasing member for biasing the swing member in one of its swing directions.
Patent Document 1: International Publication No. 2015/125222

### BRIEF SUMMARY OF THE DISCLOSURE

In recent years, there has been a demand for a moisture measuring device with improved measurement accuracy.

A moisture measuring device according to an aspect of the present disclosure includes: a biosensor having a sensor surface for acquisition of biological information; a processing unit configured to process the biological information acquired by the biosensor; a substrate having a first principal surface on which the processing unit is mounted; a housing accommodating the biosensor, the processing unit, and the substrate; and one or more attaching members disposed on the first principal surface side of the substrate to attach the substrate to the housing, wherein a total area of one or more overlap regions in which the one or more attaching members overlap the substrate in the first principal surface of the substrate when viewed in a direction perpendicular to the first principal surface of the substrate is smaller than a sensor area of the sensor surface of the biosensor.

The present disclosure can provide a moisture measuring device with improved measurement accuracy.

### BRIEF DESCRIPTION OF THE SEVERAL VIEWS OF THE DRAWINGS

FIG. 1 is a schematic perspective view of an example of a moisture measuring device according to Embodiment 1 of the present disclosure.

FIG. 2 is a schematic bottom view of an example of the moisture measuring device according to Embodiment 1 of the present disclosure.

FIG. 3 is a schematic enlarged view of a section denoted by Z1 in FIG. 2.

FIG. 4 is a block diagram illustrating the schematic configuration of an example of the moisture measuring device according to Embodiment 1 of the present disclosure.

FIG. 5 is a schematic exploded view of a sensor part and a probe part in FIG. 1.

FIG. 6 is a schematic view for explanation of the state in which a substrate is attached to a first housing.

FIG. 7 is a schematic enlarged view of a section denoted by Z2 in FIG. 6.

FIG. 8 is a plan view of the part illustrated in FIG. 6.

FIG. 9 schematically illustrates support members.

FIG. 10 is a schematic sectional view taken along line A-A in FIG. 8.

FIG. 11 schematically illustrates the configuration of a moisture measuring device according to Variation 1.

FIG. 12 schematically illustrates the configuration of a moisture measuring device according to Variation 2.

FIG. 13 is a schematic sectional view taken along line B-B in FIG. 12.

FIG. 14 schematically illustrates the configuration of a moisture measuring device according to Variation 3.

### DETAILED DESCRIPTION OF THE DISCLOSURE

### (Circumstances Leading to Present Disclosure)

Examples of known moisture measuring devices include intraoral moisture measuring devices. An intraoral moisture measuring device includes a biosensor having a sensor surface that is brought into contact with the interior of an oral cavity to acquire biological information relevant to the moisture content in the oral cavity. The intraoral moisture measuring device calculates moisture content on the basis of the acquired biological information.

The biosensor of the intraoral moisture measuring device is designed to be small enough to be inserted into an oral cavity so that the biosensor can come into contact with the interior of the oral cavity. This adds a constraint to the size of the sensor surface of the biosensor. The amount of biological information that can be acquired by the biosensor is proportional to the size of the sensor surface. That is, reducing the size of the sensor surface results in a decrease in the amount of biological information that can be acquired.

Given that such a constraint on the size of the biosensor leads to a reduction in the amount of biological information that can be acquired by the intraoral moisture measuring device, the calculation of moisture content is performed on the basis of a minute amount of biological information. When the oral cavity of the user who is the subject of measurement of moisture content is relatively dry, changes in the amount of biological information with which the calculated value of moisture content varies can be in the pico-order or nano-order. That is, the intraoral moisture measuring device performs the calculation of moisture content on the basis of a minute amount of biological information and is thus susceptible to a minute amount of noise in term of its measurement accuracy.

In a case where the biosensor is a capacitive sensor configured to measure electrostatic capacity, the noise produced in the region around a processing unit configured to process the acquired information about the electrostatic capacity measured by the biosensor makes it difficult to calculate the moisture content with high accuracy. It is therefore necessary to suppress the noise in the region around the processing unit.

The inventors conducted a thorough study and found that the noise in the region around the processing unit mounted on a substrate could be caused by the parasitic capacitance arising from the capacitive coupling between the substrate and a member in contact with the substrate. Examples of the member in contact with the substrate include an attaching member for attaching the substrate to a housing and a support member supporting the substrate in the housing.

Even if the attaching member and the support member are made of a resin material, capacitive coupling occurs, and minute parasitic capacitance arises therefrom. For example, an alternating-current component is generated when the electrostatic capacity is converted into frequency by the processing unit. The alternating-current component is likely to be polarized by the resin material and to exhibit different polarities. When the different polarities are exhibited due to polarization, the resin member and the substrate that are disposed with air therebetween constitute a capacitor structure. In this case, minute parasitic capacitance in the pico-order or nano-order can arise from the minute capacitive coupling formed between the resin material and the substrate, and as a result, noise can occur. Such a minute amount of noise in the moisture measuring device can decrease the accuracy of moisture measurement.

Accordingly, the inventors found a configuration for the noise suppression to improve the measurement accuracy, which resulted in the following disclosure.

A moisture measuring device according to an aspect of the present disclosure includes a biosensor, a processing unit, a substrate, a housing, and one or more attaching members. The biosensor has a sensor surface for acquisition of biological information. The processing unit is configured to process the biological information acquired by the biosensor. The substrate has a first principal surface on which the processing unit is mounted. The housing accommodates the biosensor, the processing unit, and the substrate. The one or more attaching members are disposed on the first principal surface side of the substrate to attach the substrate to the housing. The total area of one or more overlap regions in which the one or more attaching members overlap the substrate in the first principal surface of the substrate when viewed in a direction perpendicular to the first principal surface of the substrate is smaller than a sensor area of the sensor surface of the biosensor.

This configuration leads to an improvement in measurement accuracy.

The first principal surface of the substrate may be provided with a conductor part including a wiring pattern, and the total area of the one or more overlap regions may be smaller than the total area of the conductor part.

This configuration leads to a further improvement in measurement accuracy.

The one or more attaching members may be made of a resin.

This configuration results in the noise suppression in the one or more attaching members and leads to a further improvement in measurement accuracy.

The one or more attaching members may include one or more screws each having a screw head.

This configuration enables the substrate to be securely attached to the housing, and noise can be suppressed. This leads to an improvement in measurement accuracy.

The one or more attaching members may include one or more engagement claws, and the one or more engagement claws each may have a protrusion located in the housing, facing the first principal surface of the substrate, and protruding inward from outside the substrate.

This configuration facilitates the attaching of the substrate to the housing, and noise can be suppressed. This leads to an improvement in measurement accuracy.

The one or more engagement claws may be located apart from the first principal surface of the substrate.

This configuration reduces the possibility that the substrate is damaged when the moisture measuring device is subjected to, for example, a drop impact.

The distance between each of the one or more engagement claws and the first principal surface of the substrate may be not less than 1/10 of the thickness of the substrate and not more than ½ of the thickness of the substrate.

This configuration further reduces the possibility that the substrate is damaged when the moisture measuring device is subjected to an impact.

The substrate may have a longitudinal direction and a lateral direction, and the engagement claws may be disposed at ends on opposite sides in the lateral direction of the substrate.

This configuration enables the substrate to be securely attached to the housing and thus reduces the possibility that the substrate comes off the housing.

The engagement claws may be arranged with a spacing provided therebetween in the longitudinal direction of the substrate.

This configuration reduces the possibility that the substrate is twisted and comes off the housing due to an impact or other force exerted on the housing.

The engagement claws may include: a first engagement claw disposed between the biosensor and the processing unit in the longitudinal direction of the substrate; and a second engagement claw disposed on an opposite side from the first engagement claw in the lateral direction of the substrate and closer to the processing unit than is the first engagement claw in the longitudinal direction of the substrate.

This configuration enables the substrate to be securely attached to the housing, and noise can be further suppressed. This leads to a further improvement in measurement accuracy.

The substrate may have a second principal surface located opposite to the first principal surface, the housing may include a support member by which the second principal surface is partially supported, and a space may be defined between the housing and the substrate.

This configuration leads to a reduction in the region where capacitive coupling occurs between the housing and the substrate, and noise can be suppressed accordingly. This leads to an improvement in measurement accuracy.

The one or more attaching members may be disposed farther from the biosensor than is the processing unit.

This configuration reduces the possibility that noise comes into the processing unit from the one or more attaching members. This leads to an improvement in measurement accuracy.

The distance between each of the one or more attaching members and the processing unit may be greater than the distance between the biosensor and the processing unit.

This configuration further reduces the possibility that noise comes into the processing unit from the one or more attaching members. This leads to a further improvement in measurement accuracy.

The biosensor may be a capacitive sensor configured to measure electrostatic capacity, and the processing unit may be configured to perform conversion processing in which the electrostatic capacity measured by the capacitive sensor is converted into frequency.

This configuration leads to a further improvement in measurement accuracy.

An embodiment of the present disclosure will be described below with reference to the accompanying drawings. Note that the following description is merely illustrative in nature and is not intended to limit the present disclosure, articles to which it is applied, or its use. The accompanying drawings are schematic, and the dimension ratios of constituent elements in the drawings do not necessarily fully correspond to the actual dimension ratios.

The terms “first”, “second”, “third”, “fourth”, and so on in the following description are added for convenience to make constituent components distinguishable from one another and are not intended to be limiting.

### Embodiment 1

### [Overall Configuration]

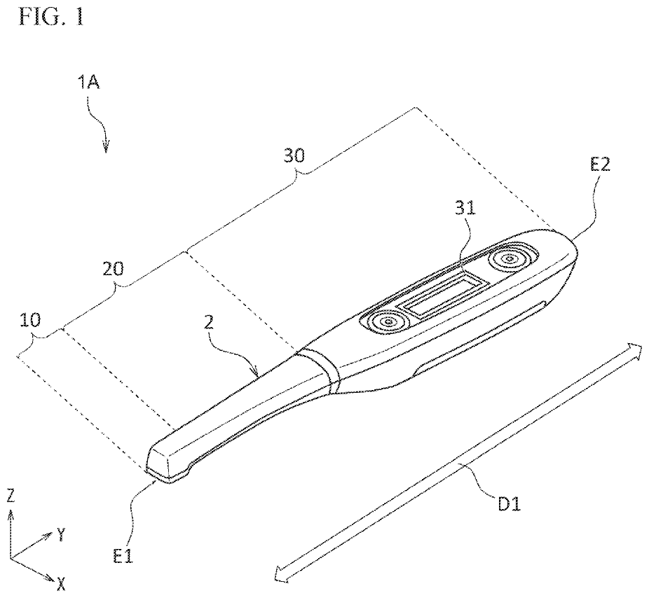

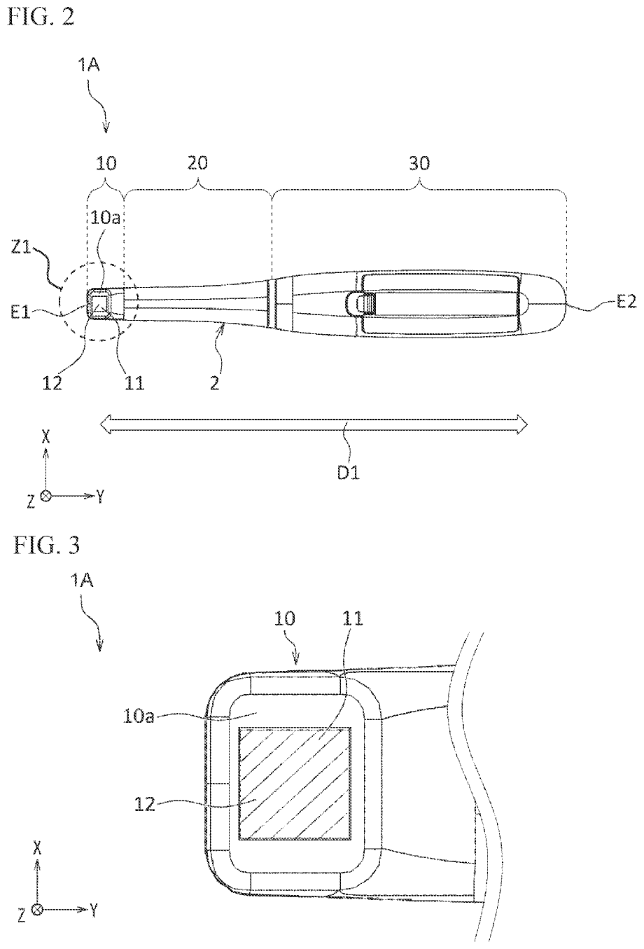

FIG. 1 is a schematic perspective view of an example of a moisture measuring device 1A according to Embodiment 1 of the present disclosure. FIG. 2 is a schematic bottom view of an example of the moisture measuring device 1A according to Embodiment 1 of the present disclosure. FIG. 3 is a schematic enlarged view of a section denoted by Z1 in FIG. 2. The X, Y, and Z directions in the drawings denote the width direction, the length direction, and the height direction, respectively, of the moisture measuring device 1A. The hatched area in FIG. 3 denotes a sensor surface 12.

The following describes Embodiment 1, in which the moisture measuring device 1A is an example of a moisture measuring device (an intraoral moisture checker) configured to measure the moisture content in the oral cavity.

### <Exterior Appearance>

The following describes the exterior appearance of the moisture measuring device 1A. Referring to FIGS. 1 and 2, the moisture measuring device 1A includes a housing 2.

The housing 2 has a rod shape with a longitudinal direction D1. Specifically, the housing 2 includes a sensor part 10, a probe part 20, and a grip part 30.

The sensor part 10 is designed to come into contact with a measurement site of a living body. For example, the measurement site of the living body is a body part in an oral cavity. A tongue is an example of the body part in the oral cavity. The sensor part 10 is located at one end E1, which is one of two opposite ends in the longitudinal direction D1 of the moisture measuring device 1A. The sensor part 10 is designed with its outer dimensions less than those of the probe part 20 and the grip part 30. For example, the sensor part 10 is designed to be smaller in dimension than the probe part 20 and the grip part 30 in each of the X and Y directions.

As illustrated in FIGS. 2 and 3, the sensor part 10 has a contact surface 10a, which is designed to come into contact with the measurement site of a living body. The contact surface 10a is located on the end E1 side in the longitudinal direction D1 of the housing 2 and extends in directions (the X and Y directions) that form an angle with an end face on the end E1 side. The sensor surface 12, which is a surface of a biosensor 11, is exposed at the contact surface 10a.

The probe part 20 forms a connection between the sensor part 10 and the grip part 30. The probe part 20 is in the form of a rod. The dimensions in the X and Z directions of the probe part 20 extending from the grip part 30 each become smaller toward the sensor part 10. In other words, the probe part 20 extending from the grip part 30 tapers to the sensor part 10.

The grip part 30 is designed to be gripped by the user. The grip part 30 is located at the other end E2, which is the other end in the longitudinal direction D1 of the moisture measuring device 1A. The grip part 30 is in the form of a rod. The grip part 30 is designed with its outer dimensions greater than those of the sensor part 10 and the probe part 20. For example, the grip part 30 is designed to be greater in dimension than the sensor part 10 and the probe part 20 in each of the X, Y, and Z directions.

The housing 2 is made of, for example, a resin. Part of the housing 2 may be made of a metallic material.

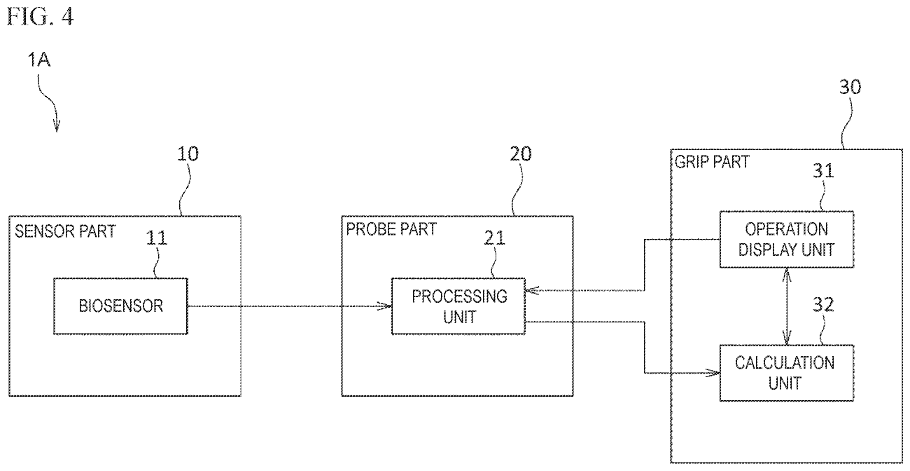

The following describes functional modules of the moisture measuring device 1A. FIG. 4 is a block diagram illustrating the schematic configuration of an example of the moisture measuring device 1A according to Embodiment 1 of the present disclosure. Referring to FIG. 4, the moisture measuring device 1A includes the biosensor 11, a processing unit 21, an operation display unit 31, and a calculation unit 32.

Embodiment 1 is not limited to the following example in which the moisture measuring device 1A includes the operation display unit 31 and the calculation unit 32. That is, the operation display unit 31 and the calculation unit 32 are optional and may be included in a device other than the moisture measuring device 1A.

### <Biosensor>

The biosensor 11 acquires biological information. The term “biological information” refers to various kinds of physiological and anatomical information emanating from a living body. Examples of the biological information include electrostatic capacity, resistance, and impedance. The biosensor 11 is brought into contact with a measurement site of the living body and acquires biological information of the measurement site in contact with the biosensor 11.

The biosensor 11 in Embodiment 1 is a capacitive sensor. The biosensor 11 is brought into contact with a measurement site in an oral cavity and acquires information about electrostatic capacity. The biological information acquired by the biosensor 11 in Embodiment 1 is information about electrostatic capacity.

As illustrated in FIG. 3, the biosensor 11 is disposed in the sensor part 10. Specifically, the biosensor 11 is disposed in the contact surface 10a of the sensor part 10. For example, the biosensor 11 is fitted in a recess on the contact surface 10a side of the sensor part 10 of the housing 2.

The biosensor 11 is planar. Specifically, the biosensor 11 has the sensor surface 12 for acquisition of biological information. The sensor surface 12 is exposed on the contact surface 10a side of the sensor part 10. For example, the sensor surface 12 is rectangular in shape when viewed in the height direction (the Z direction) of the moisture measuring device 1A. The biological information is captured through the sensor surface 12 in contact with the measurement site. That is, the biosensor 11 acquires the biological information with the sensor surface 12 being in contact with the measurement site.

The biological information acquired by the biosensor 11 is transmitted to the processing unit 21.

### <Processing Unit>

The processing unit 21 processes the biological information acquired by the biosensor 11. Specifically, the processing unit 21 subjects the biological information acquired by the biosensor 11 to conversion processing and then outputs the resultant information.

The processing unit 21 converts the information acquired by the biosensor 11 from analog to digital form. The processing unit 21 in Embodiment 1 includes a frequency conversion circuit in which the information acquired by the biosensor 11 and concerning electrostatic capacity is converted into frequency. For example, the biosensor 11 regarded as electrostatic capacity is subjected to charge and discharge cycles by the processing unit 21, which then converts the electrostatic capacity into the frequency calculated from the period determined on the basis of the charge-discharge speed. Thus, the output value obtained from the biosensor 11 is outputted as a frequency value by the processing unit 21.

The processing unit 21 may be implemented by a semiconductor device. The processing unit 21 may, for example, be a microcomputer, a CPU, an MPU, a GPU, a DSP, an FPGA, an ASIC, a discrete semiconductor, or an LSI. The functions of the processing unit 21 may be achieved by hardware alone or a combination of hardware and software. The processing unit 21 performs predetermined functions by executing arithmetic processing based on data and programs retrieved from storage (not illustrated) in the processing unit 21. The storage may, for example, be a hard disk drive (HDD), an SSD, RAM, DRAM, ferroelectric memory, flash memory, a magnetic disk, or a combination of two or more of these.

The processing unit 21 subjects the biological information acquired by the biosensor 11 to conversion processing and then stores the resultant information in the storage. After storing the information in the storage, the processing unit 21 transmits the stored information to the calculation unit 32. For example, the processing unit 21 transmits the information to the calculation unit 32 on the basis of trigger information for measurement initiation. The trigger information for measurement initiation may, for example, be generated on the basis of information inputted to the operation display unit 31.

The processing unit 21 is disposed in the probe part 20. Specifically, the processing unit 21 is mounted on a substrate held within the probe part 20. The substrate will be described later. This layout enables the distance between the processing unit 21 and the biosensor 11 to be shortened. Accordingly, noise can be suppressed.

### <Operation Display Unit>

The operation display unit 31 accepts inputs from the user and displays information about moisture content. For example, the operation display unit 31 includes an operation part and a display part. The operation part accepts an operation performed by the user, and the display part displays information.

The operation part includes one or more buttons for accepting inputs from the user. The buttons include for example, a power button for turning on and turning off of power supply.

The display part displays information about moisture content. For example, the display part is a display. The information about moisture content is transmitted to the display part from, for example, the calculation unit 32.

The operation display unit 31 is disposed on an upper surface of the grip part 30.

### <Calculation Unit>

The calculation unit 32 calculates the moisture content on the basis of the information processed by the processing unit 21. Specifically, the calculation unit 32 receives, from the processing unit 21, information derived from the biological information through conversion processing and then calculates the moisture content on the basis of the information that has undergone conversion processing. The calculation unit 32 in Embodiment 1 receives, from the processing unit 21, the information obtained through the conversion from electrostatic capacity into frequency and then determines the moisture content on the basis of the information about frequency. The information about the moisture content calculated by the calculation unit 32 is transmitted to the operation display unit 31.

The calculation unit 32 may be implemented by a semiconductor device. The calculation unit 32 may, for example, be a microcomputer, a CPU, an MPU, a GPU, a DSP, an FPGA, an ASIC, a discrete semiconductor, or an LSI. The functions of the calculation unit 32 may be achieved by hardware alone or a combination of hardware and software. The calculation unit 32 performs predetermined functions by executing arithmetic processing based on data and programs retrieved from storage (not illustrated) in the processing unit 21.

The calculation unit 32 is disposed in the grip part 30.

The moisture measuring device 1A includes a control unit that performs centralized control of the individual elements of the moisture measuring device 1A. The control unit includes, for example, memory and a processing circuit. Programs are stored in the memory. The processing circuit is provided for a processor, such as a central processing unit (CPU). For example, the processor included in the control unit executes programs stored in the memory. The control unit in Embodiment 1 controls the biosensor 11, the processing unit 21, the operation display unit 31, and the calculation unit 32.

### [Internal Configuration of Sensor Part and Probe Part]

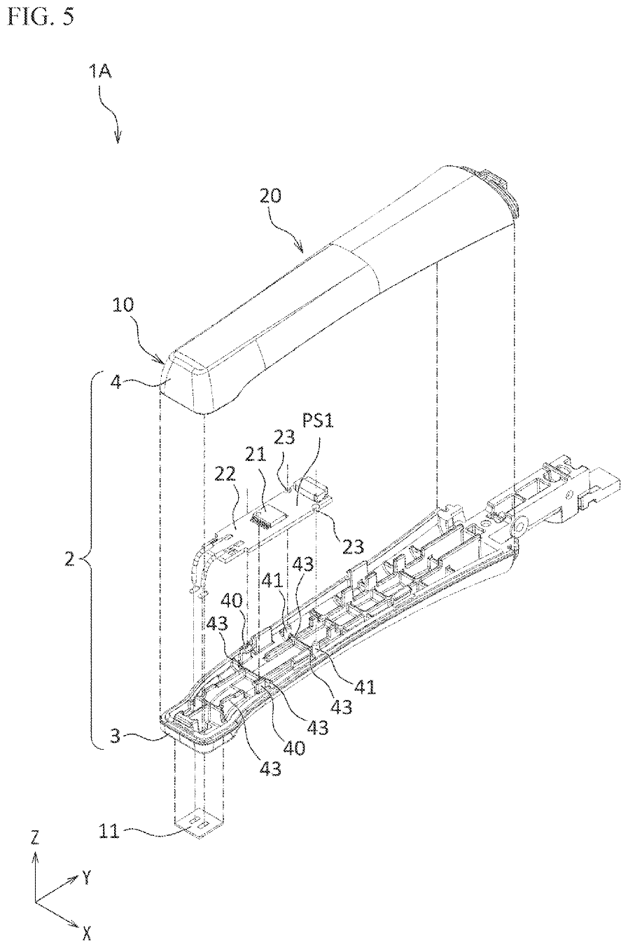

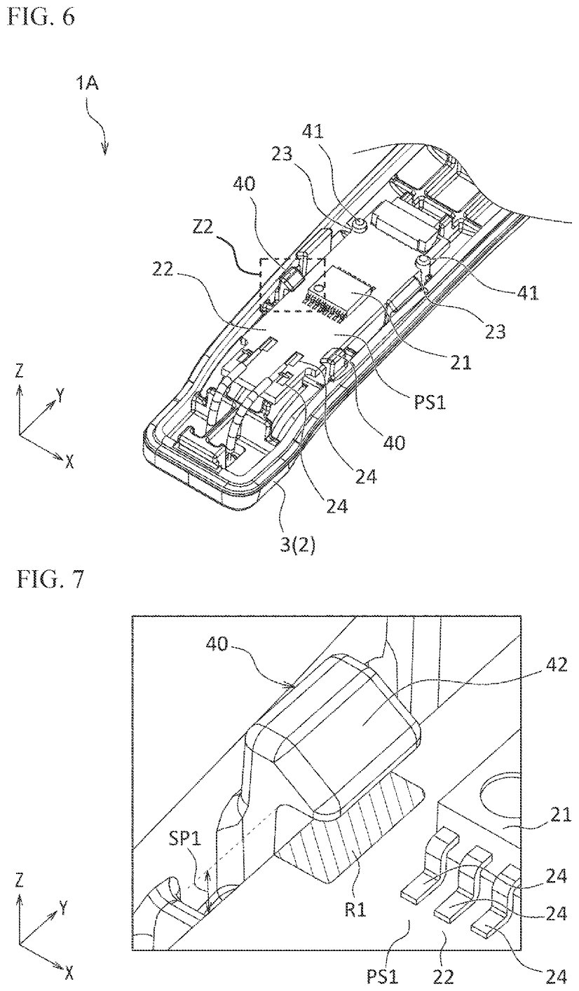

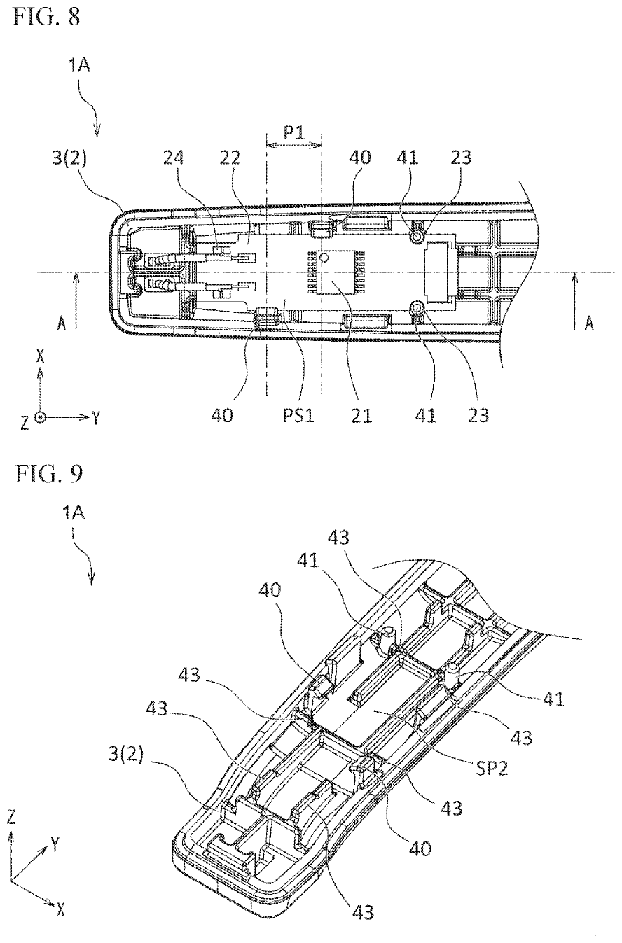

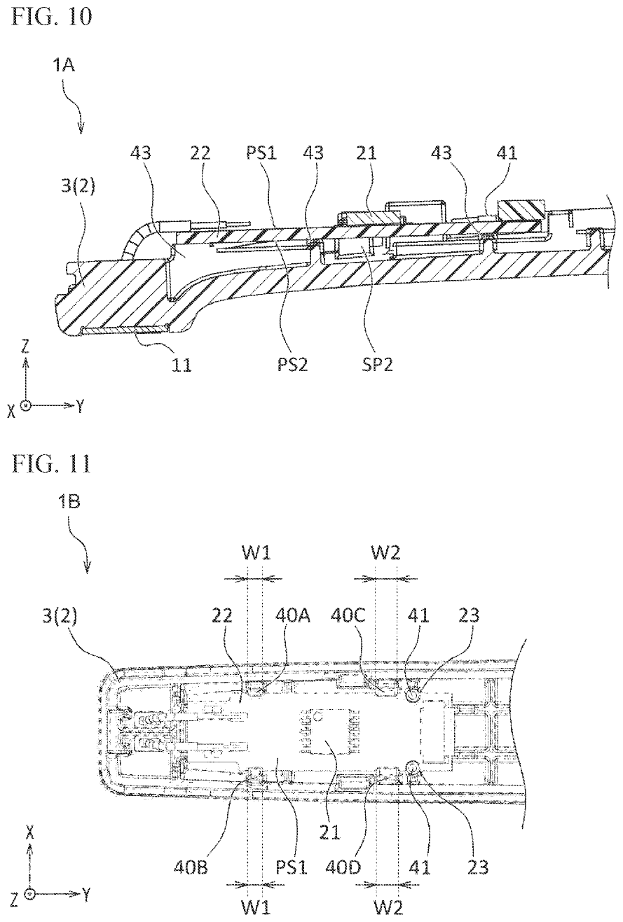

FIG. 5 is a schematic exploded view of the sensor part 10 and the probe part 20 in FIG. 1. FIG. 6 is a schematic view for explanation of the state in which a substrate 22 is attached to a first housing 3. FIG. 7 is a schematic enlarged view of a section denoted by Z2 in FIG. 6. FIG. 8 is a plan view of the part illustrated in FIG. 6. FIG. 9 schematically illustrates support members 43. FIG. 10 is a schematic sectional view taken along line A-A in FIG. 8.

As illustrated in FIGS. 5 to 8, the housing 2, the biosensor 11, the processing unit 21, and the substrate 22 constitute the sensor part 10 and the probe part 20 of the moisture measuring device 1A. The housing 2 includes a first housing 3 and a second housing 4 connected to the first housing 3. The first housing 3 is a housing in which the biosensor 11 is disposed. The second housing 4 is a housing opposite to the housing in which the biosensor 11 is disposed. For example, the first housing 3 and the second housing 4 are connected together by fitting.

The substrate 22 is in the form of a plate having a longitudinal direction (the Y direction) and a lateral direction (the X direction). The substrate 22 has a first principal surface PS1, which is oriented toward the second housing 4. The first principal surface PS1 is a mounting place for components (e.g., the processing unit 21), an IC, a connector, a wiring pattern, and/or an electrode. The wiring pattern and/or the electrode is herein referred to as a conductor part 24.

The substrate 22 has cutouts 23, which are provided in the respective ends on the opposite sides in the lateral direction (the X direction). For example, the cutouts 23 are each semicircular in shape when viewed in a direction perpendicular to the first principal surface PS1 of the substrate 22. Positioning pins 41, which will be described later, are fitted in the cutouts 23. The substrate 22 in Embodiment 1 has two cutouts 23, which are provided in the respective ends on the opposite sides in the lateral direction (the X direction). The two cutouts 23 are disposed farther from the biosensor 11 than is the processing unit 21 when viewed in the direction perpendicular to the first principal surface PS1 of the substrate 22.

The substrate 22 is disposed in the housing 2. Specifically, the substrate 22 is located in the probe part 20 and is attached to the first housing 3.

The first housing 3 includes attaching members 40. The substrate 22 is attached to the housing 2 with the attaching members 40. Specifically, the attaching members 40 are disposed on the first principal surface PS1 side of the substrate 22, as illustrated in FIGS. 6 and 7. The attaching members 40 restrict the movement of the substrate 22 in the thickness direction (the Z direction) of the substrate 22. That is, the substrate 22 is held in the housing 2 by the attaching members 40.

The attaching members 40 in Embodiment 1 are engagement claws. The engagement claws each have a protrusion 42, which is located in the housing 2. The protrusions 42 face the first principal surface PS1 of the substrate 22 and protrude inward from outside the substrate 22. The protrusions 42 overlap the substrate 22 when viewed in the direction (the Z direction) perpendicular to the first principal surface PS1 of the substrate 22. Thus, the first principal surface PS1 of the substrate 22 comes into contact with the protrusions 42 when the substrate 22 moves in the thickness direction (the Z direction) of the substrate 22. As a result, the movement of the substrate 22 in the thickness direction (the Z direction) of the substrate 22 is restricted by the protrusions 42. In this state, the substrate 22 is attached to the first housing 3. The engagement claws may be hereinafter also denoted by 40.

As illustrated in FIG. 7, the engagement claws 40 are located apart from the first principal surface PS1 of the substrate 22 in the direction (the Z direction) perpendicular to the first principal surface PS1 of the substrate 22. The distance between each of the engagement claws 40 and the first principal surface PS1 of the substrate 22 is not less than 0.1 times the thickness of the substrate 22 and is not more than 0.5 times the thickness of the substrate 22. Specifically, a clearance SP1 is left between the protrusion 42 of each engagement claw 40 and the first principal surface PS1 of the substrate 22. The clearance SP1 is smaller than the thickness of the substrate 22. For example, the clearance SP1 is not less than 0.1 times the thickness of the substrate 22 and is not more than 0.5 times the thickness of the substrate 22. This reduces the possibility that the substrate 22 is damaged when the moisture measuring device 1A is subjected to, for example, a drop impact.

The first housing 3 in Embodiment 1 includes two attaching members 40. As illustrated in FIG. 8, the two attaching members 40 are disposed at the respective ends on the opposite sides in the lateral direction (the X direction) of the substrate 22. The two attaching members 40 are arranged with a spacing (P1) provided therebetween in the longitudinal direction (the Y direction) of the substrate 22 when viewed in the direction perpendicular to the first principal surface PS1 of the substrate 22. The two attaching members 40 are staggered apart in the longitudinal direction (the Y direction) of the substrate 22. This reduces the possibility that the substrate 22 is twisted and comes off the attaching members 40 when the moisture measuring device 1A is subjected to, for example, a drop impact.

Specifically, a first engagement claw 40 and a second engagement claw 40 are disposed at the respective ends on the opposite sides in the lateral direction (the X direction) of the substrate 22. The first engagement claw 40 is located between the biosensor 11 and the processing unit 21 when viewed in the direction perpendicular to the first principal surface PS1 of the substrate 22. The second engagement claw 40 is disposed on an opposite side from the first engagement claw 40 in the lateral direction of the substrate 22. The second engagement claw 40 is disposed closer to the processing unit 21 than is the first engagement claw 40 in the longitudinal direction of the substrate 22.

As illustrated in FIGS. 6 and 8, the first housing 3 includes the positioning pins 41, which protrude from the inside of the first housing 3 and extend toward the substrate 22. The substrate 22 is positioned on the first housing 3, with the positioning pins 41 being fitted in the cutouts 23 of the substrate 22. The positioning pins 41 restrict the movement of the substrate 22 in the directions (the X direction and the Y direction) perpendicular to the thickness direction of the substrate 22. For example, the positioning pins 41 are each in the form of a circular column.

The first housing 3 in Embodiment 1 includes two positioning pins 41.

As illustrated in FIGS. 9 and 10, the first housing 3 has more than one support member 43 supporting the substrate 22. A second principal surface PS2 of the substrate 22 is located opposite to the first principal surface PS1 and is partially supported by the support members 43.

The expression “partially supported” herein means that the second principal surface PS2 of the substrate 22 is supported in part but not in whole. For example, the support members 43 support two end portions on the opposite sides in the longitudinal direction (the Y direction) of the substrate 22 and also support, in the midsection in the longitudinal direction (the Y direction) of the substrate 22, and two end portions on the opposite sides in the lateral direction (the X direction) of the substrate 22 when viewed in the direction (the Z direction) perpendicular to the second principal surface PS2 of the substrate 22.

The support members 43 protrude from an inner wall of the first housing 3 and extend toward the second principal surface PS2 of the substrate 22. The support members 43 are, for example, each plate-like in shape. For example, the support members 43 are each substantially equal in thickness to, for example, the substrate 22.

The second principal surface PS2 of the substrate 22 is partially supported by the support members 43 such that a space SP2 is defined between the second principal surface PS2 of the substrate 22 and the first housing 3. In the event that, for example, an impact is exerted on the moisture measuring device 1A, the force of the impact can be dissipated into the support members 43. This is due to the space SP2 between the second principal surface PS2 of the substrate 22 and the first housing 3.

### [Configuration for Noise Suppression]

The following describes a configuration for noise suppression in the moisture measuring device 1A.

The total area of overlap regions R1 in which the attaching members 40 overlap the substrate 22 when the moisture measuring device 1A is viewed in the direction (the Z direction) perpendicular to the first principal surface PS1 of the substrate 22 is smaller than the sensor area of the sensor surface 12 of the biosensor 11.

As illustrated in FIG. 7, “overlap region R1” refers to regions in which the protrusions 42 of the attaching members 40 overlap the substrate 22 in the first principal surface PS1 of the substrate 22 when viewed in the direction (the Z direction) perpendicular to the first principal surface PS1 of the substrate 22. In other words, “overlap region R1” refers to regions of the attaching members 40 projected onto the first principal surface PS1 of the substrate 22 when viewed in the direction (the Z direction) perpendicular to the first principal surface PS1 of the substrate 22.

“The total area of the overlap regions R1” refers to the sum of the areas of the overlap regions R1 defined by the attaching members 40 in the first principal surface PS1 of the substrate 22. In Embodiment 1, two overlap regions R1 are defined since the first housing 3 has two attaching members 40. Thus, the sum of the areas of the two overlap regions R1 is the total area of the overlap regions R1.

“The sensor area of the sensor surface 12” refers to the surface area of the sensor surface 12 when viewed in the direction (the Z direction) perpendicular to the sensor surface 12 as in FIG. 3. The area of the sensor surface 12 in Embodiment 1 is, for example, approximately 50 mm².

The total area of the overlap regions R1 in the moisture measuring device 1A is smaller than the sensor area of the sensor surface 12 of the biosensor 11. This configuration leads to a reduction in the region where capacitive coupling occurs between the attaching members 40 and the substrate 22. The parasitic capacitance arising from the capacitive coupling is reduced accordingly.

The total area of the overlap regions R1 may be smaller than the total area of the conductor part 24 on the first principal surface PS1 of the substrate. The conductor part 24 on the substrate 22 is made of a metallic material and includes, for example, the wiring pattern and/or the electrode on the first principal surface PS1 of the substrate 22. “The total area of the conductor part 24” refers to the sum of the areas of conductors including the wiring pattern and/or the electrode on the first principal surface PS1 of the substrate 22 when viewed in the direction (the Z direction) perpendicular to the first principal surface PS1 of the substrate 22.

For example, the total area of the overlap regions R1 is equal to or more than 0.01 times the sensor area of the sensor surface 12 and is less than one times the sensor area of the sensor surface 12.

The clearance SP1 is left between the protrusion 42 of each attaching member 40 and the first principal surface PS1 of the substrate 22. Keeping the attaching members 40 apart from the substrate 22 reduces the possibility of capacitive coupling.

The second principal surface PS2 side of the substrate 22 is partially supported by the support members 43, with the space SP2 being defined between the second principal surface PS2 of the substrate 22 and the first housing 3.

Providing the support members 43 that support part of the second principal surface PS2 of the substrate 22 results in a reduction in the area of contact between the first housing 3 and the substrate 22. This leads to a reduction in the region where capacitive coupling occurs between the first housing 3 and the substrate 22.

The support members 43 do not overlap the processing unit 21 when viewed in the direction (the Z direction) perpendicular to the second principal surface PS2 of the substrate 22. The processing unit 21 in Embodiment 1 is disposed at or close to the midpoint in the longitudinal direction (the Y direction) of the substrate 22. Given this layout, the support members 43 support the two end portions on the opposite sides in the longitudinal direction (the Y direction) of the substrate 22 and also support, in the midsection in the longitudinal direction (the Y direction) of the substrate 22, the two end portions on the opposite sides in the lateral direction (the X direction) of the substrate 22 when viewed in the direction (the Z direction) perpendicular to the second principal surface PS2 of the substrate 22. This configuration reduces the possibility that capacitive coupling occurs in the region around the processing unit 21. This results in the suppression of parasitic capacitance.

The attaching member 40, the positioning pins 41, and the support members 43 are each made of a resin material. The parasitic capacitance arising from capacitive coupling may be lower than a case where the attaching member 40, the positioning pins 41, and the support members 43 are each made of a metallic material. Accordingly, noise can be suppressed.

### [Effects]

The moisture measuring device 1A according to Embodiment 1 produces the following effects.

The moisture measuring device 1A includes the biosensor 11, the processing unit 21, the substrate 22, the housing 2, and the attaching members 40. Specifically, the biosensor 11 has the sensor surface 12 for acquisition of biological information. The processing unit 21 is configured to process the biological information acquired by the biosensor 11. The substrate 22 has the first principal surface PS1, on which the processing unit 21 is mounted. The housing 2 accommodates the biosensor 11, the processing unit 21, and the substrate 22. The attaching members 40 are disposed on the first principal surface PS1 side of the substrate 22 to attach the substrate 22 to the housing 2. The total area of the overlap regions R1 in which the attaching members 40 overlap the substrate 22 in the first principal surface PS1 of the substrate 22 when viewed in the direction (the Z direction) perpendicular to the first principal surface PS1 of the substrate 22 is smaller than the sensor area of the sensor surface 12 of the biosensor 11.

This leads to an improvement in the accuracy of moisture measurement. Specifically, the region where capacitive coupling occurs between the attaching members 40 and the substrate 22 is reduced, and the parasitic capacitance between the attaching members 40 and the substrate 22 is reduced accordingly. This results in the noise suppression in the region around the processing unit 21 and, by extension, reduces the degree to which the data acquired by the biosensor 11 is affected by noise. As a result, the measurement accuracy of the moisture measuring device 1A is improved.

The first principal surface PS1 of the substrate 22 of the moisture measuring device 1A is provided with the conductor part 24 including the wiring pattern, and the total area of the overlap regions R1 is smaller than the total area of the conductor part 24. This leads to a further reduction in the parasitic capacitance between the attaching members 40 and the substrate 22 such that noise can be further suppressed. As a result, the measurement accuracy of the moisture measuring device 1A can be further improved.

The attaching members 40 are made of a resin. The possibility of the capacitive coupling between the attaching members 40 and the substrate 22 is lower than a case where the attaching members 40 are made of a metallic material. As a result, noise can be further suppressed, and the measurement accuracy of the moisture measuring device 1A can be further improved.

The attaching members 40 include the engagement claws 40. The engagement claws 40 have the respective protrusions 42 located in the housing 2, facing the first principal surface PS1 of the substrate 22, and protruding inward from outside the substrate 22. The substrate 22 can thus be easily attached to the housing 2, and the overlap regions R1 can be reduced in size. This leads to a further reduction in the parasitic capacitance between the attaching members 40 and the substrate 22 such that noise can be further suppressed. As a result, the measurement accuracy of the moisture measuring device 1A can be further improved.

The substrate 22 has a longitudinal direction (the Y direction) and a lateral direction (the X direction). The engagement claws 40 are disposed at the respective ends on the opposite sides in the lateral direction of the substrate 22. This configuration reduces the possibility that the substrate 22 comes off the housing 2.

The engagement claws 40 are arranged with the spacing P1 provided therebetween in the longitudinal direction (the Y direction) of the substrate 22. This configuration reduces the possibility that the substrate 22 is twisted and comes off the attaching members 40 when the moisture measuring device 1A is subjected to, for example, a drop impact.

The substrate 22 has the second principal surface PS2 located opposite to the first principal surface PS1. The housing 2 includes the support members 43 by which the second principal surface PS2 is partially supported. The space SP2 is defined between the housing 2 and the substrate 22. This configuration results in a reduction in the area of contact between the housing 2 and the substrate 22. This leads to a reduction in the region where capacitive coupling occurs between the housing 2 and the substrate 22. The parasitic capacitance arising from the capacitive coupling can be reduced accordingly. As a result, noise can be suppressed, and the measurement accuracy of the moisture measuring device 1A is improved.

The biosensor 11 is a capacitive sensor configured to measure electrostatic capacity. The processing unit 21 performs conversion processing in which the electrostatic capacity measured by the capacitive sensor is converted into frequency. This configuration leads to an improvement in the measurement accuracy of the moisture measuring device 1A.

Embodiment 1 is not limited to the aforementioned example in which the moisture measuring device 1A includes functional modules such as the biosensor 11, the processing unit 21, the operation display unit 31, and the calculation unit 32. For example, the processing unit 21 and the operation display unit 31 may be combined in one unit. Alternatively, the biosensor 11 and the processing unit 21 may be combined in one unit.

Embodiment 1 is not limited to the aforementioned example in which the operation display unit 31 and the calculation unit 32 are included in the moisture measuring device 1A. It is not required that the operation display unit 31 and/or the calculation unit 32 be included in the moisture measuring device 1A. The operation display unit 31 and/or the calculation unit 32 may be included in a device other than the moisture measuring device 1A. The operation display unit 31 and/or the calculation unit 32 may, for example, be implemented by a smartphone. In this case, the moisture measuring device 1A may include a communication unit capable of wireless communication.

Embodiment 1 is not limited to the aforementioned example in which the housing 2 includes the sensor part 10, the probe part 20, and the grip part 30. The housing 2 may have any shape having a longitudinal direction.

Embodiment 1 is not limited to the aforementioned example in which the biosensor 11 is a capacitive sensor. The biosensor 11 may be any sensor capable of acquiring biological information.

Embodiment 1 is not limited to the aforementioned example in which the sensor surface 12 of the biosensor 11 is rectangular in shape when viewed in the height direction (the Z direction) of the moisture measuring device 1A. For example, the sensor surface 12 of the biosensor 11 may be polygonal, circular, or elliptical when viewed in the height direction (the Z direction) of the moisture measuring device 1A.

Embodiment 1 is not limited to the aforementioned example in which the processing unit 21 includes a conversion circuit for conversion processing in which electrostatic capacity is converted into frequency. The processing unit 21 may include a circuit in which the biological information acquired by the biosensor 11 is converted into information other than frequency.

Embodiment 1 is not limited to the aforementioned example in which the processing unit 21 is disposed at or close to the midpoint in the longitudinal direction (the Y direction) of the substrate 22. For example, the processing unit 21 may be disposed closer to the biosensor 11. Noise is less likely to be introduced into the processing unit 21 due to the shorter distance between the processing unit 21 and the biosensor 11.

Embodiment 1 is not limited to the aforementioned example in which the operation display unit 31 includes the operation part and the display part. It is required that at least one of the operation part and the display part be included in the operation display unit 31.

Embodiment 1 is not limited to the aforementioned example in which the housing 2 includes two attaching members 40. The housing 2 may include one or more attaching members 40.

Embodiment 1 is not limited to the aforementioned example in which the attaching members 40 are engagement claws. Each of the attaching members 40 may be any member that can be disposed on the first principal surface PS1 side of the substrate 22 to attach the substrate 22 to the housing 2.

Embodiment 1 is not limited to the aforementioned example in which the attaching members 40 are disposed at the respective ends on the opposite sides in the lateral direction (the X direction) of the substrate 22. The attaching members 40 may be disposed on the respective end portions on the opposite sides in the longitudinal direction (the Y direction) of the substrate 22.

Embodiment 1 is not limited to the aforementioned example in which the clearance SP1 is defined between the protrusion 42 of each attaching member 40 and the first principal surface PS1 of the substrate 22. For example, the protrusion 42 may be in contact with the first principal surface PS1 of the substrate 22, without the clearance SP1 therebetween.

Embodiment 1 is not limited to the aforementioned example in which the housing 2 includes two positioning pins 41. For example, the housing 2 may include one or more positioning pins 41. The positioning pins 41 of the housing 2 are optional.

Embodiment 1 is not limited to the aforementioned example in which the housing 2 includes more than one support member 43. The housing 2 may include one or more support members 43. For example, one support member 43 in the form of a frame may extend along the periphery of the second principal surface PS2 of the substrate 22.

Embodiment 1 is not limited to the aforementioned example in which the attaching members 40, the positioning pins 41, and the support members 43 are each made of a resin material. For example, the attaching member 40, the positioning pins 41, and the support members 43 may each be made of a metallic material.

Embodiment 1 is not limited to the aforementioned example in which the moisture measuring device 1A is an intraoral moisture measuring device (an oral moisture checker) configured to measure the moisture content in the oral cavity. For example, the moisture measuring device 1A may be a skin moisture measuring device configured to measure the moisture content of the skin.

### [Variation 1]

FIG. 11 schematically illustrates the configuration of a moisture measuring device 1B according to Variation 1. As illustrated in FIG. 11, the housing 2 of the moisture measuring device 1B may include four attaching members 40A, 40B, 40C, and 40D. The attaching members 40A, 40B, 40C, and 40D in Variation 1 are hereinafter referred to as a first attaching member 40A, a second attaching member 40B, a third attaching member 40C, and a fourth attaching member 40D, respectively. The other configuration of the moisture measuring device 1B according to Variation 1 is identical to that of the moisture measuring device 1A according to Embodiment 1.

When viewed in the direction (the Z direction) perpendicular to the first principal surface PS1 of the substrate 22, the first attaching member 40A and the second attaching member 40B are disposed between the biosensor 11 and the processing unit 21, with the third attaching member 40C and the fourth attaching member 40D being disposed farther from the biosensor 11 than is the processing unit 21. The first attaching member 40A and the second attaching member 40B face each other in the lateral direction (the X direction) of the substrate 22, and the third attaching member 40C and the fourth attaching member 40D face each other in the lateral direction (the X direction) of the substrate 22.

Thus, the substrate 22 of the moisture measuring device 1B can be more securely attached to the housing 2.

The first attaching member 40A and the second attaching member 40B are smaller in size than the third attaching member 40C and the fourth attaching member 40D. Specifically, the width W1 of each of the first attaching member 40A and the second attaching member 40B is less than the width W2 of the third attaching member 40C and the fourth attaching member 40D when viewed in the direction (the Z direction) perpendicular to the first principal surface PS1 of the substrate 22. The widths W1 and W2 each refer to the dimension in the longitudinal direction (the Y direction) of the substrate 22.

Accordingly, the capacitance between the biosensor 11 and the processing unit 21 is reduced such that noise can be further suppressed.

Variation 1 is not limited to the aforementioned example in which the housing 2 includes the four attaching members 40A to 40D. For example, the housing 2 may include three attaching members. In this case, the first attaching member 40A may be eliminated; that is, the housing 2 may include the second to fourth attaching members 40B to 40D. Alternatively, the first attaching member 40A and the fourth attaching member 40D may be eliminated; that is, the housing 2 may include the second attaching member 40B and the third attaching member 40C.

### [Variation 2]

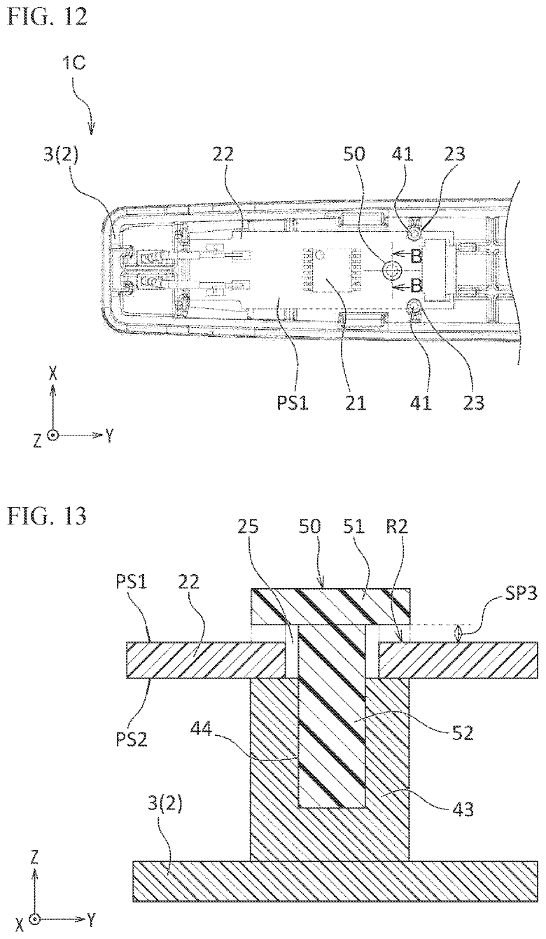

FIG. 12 schematically illustrates the configuration of a moisture measuring device 1C according to Variation 2. FIG. 13 is a schematic sectional view taken along line B-B in FIG. 12. As illustrated in FIGS. 12 and 13, an attaching member 50 may be a screw. The other configuration of the moisture measuring device 1C according to Variation 2 is identical to that of the moisture measuring device 1A according to Embodiment 1.

The attaching member 50 includes a screw head 51 and a screw main body 52. The screw main body 52 is connected to the screw head 51. The screw head 51 is discoid. The screw main body 52 is in the form of a circular column smaller in diameter than the screw head 51 and has an external thread extending along its outer wall. The attaching member 50 is made of, for example, a resin material.

When viewed in the direction (the Z direction) perpendicular to the first principal surface PS1 of the substrate 22, the attaching member 50 is disposed farther from the biosensor 11 than is the processing unit 21 in the longitudinal direction (the Y direction) of the substrate 22. The attaching member 50 is located at the midpoint in the lateral direction (the X direction) of the substrate 22.

The substrate 22 has a through-hole 25, into which the attaching member 50 is fitted. The outer diameter of the through-hole 25 is larger than the outer diameter of the screw main body 52 and is smaller than the outer diameter of the screw head 51.

The support member 43 is located in the fitting position of the attaching member 50 when viewed in the direction (the Z direction) perpendicular to the first principal surface PS1 of the substrate 22. The support member 43 has a threaded hole 44, into which the screw main body 52 is rotated. The screw main body 52 of the attaching member 50 is inserted into the through-hole 25 of the substrate 22 and is rotated into the threaded hole 44. Accordingly, the attaching member 50 restricts the movement of the substrate 22 in the thickness direction (the Z direction) of the substrate 22.

A clearance SP3 is left between the screw head 51 of the attaching member 50 and the first principal surface PS1 of the substrate 22. The clearance SP3 is substantially equal in size to the clearance SP1. Creating the clearance SP3 between the screw head 51 and the first principal surface PS1 of the substrate 22 reduces the possibility that the substrate 22 is damaged when the moisture measuring device 1C is subjected to, for example, a drop impact. The screw head 51 and the substrate 22 are kept from being capacitively coupled to each other. Accordingly, noise can be suppressed.

An overlap region R2 in the moisture measuring device 1C is a region in which the screw head 51 overlaps the substrate 22 when viewed in the direction (the Z direction) perpendicular to the first principal surface PS1 of the substrate 22. In other words, the overlap region R2 is a region of the screw head 51 projected onto the first principal surface PS1 of the substrate 22 when viewed in the direction (the Z direction) perpendicular to the first principal surface PS1 of the substrate 22.

The total area of the overlap region R2 in the moisture measuring device 1C is smaller than the sensor area of the sensor surface 12 of the biosensor 11. Accordingly, noise can be suppressed. As a result, the measurement accuracy of the moisture measuring device 1C can be improved. The substrate 22 can be more securely attached to the housing 2 with the attaching member 50 that is in the form of a screw.

Variation 2 is not limited to the aforementioned example in which the housing 2 includes one attaching member 50. The housing 2 may include one or more attaching members 50. For example, two attaching members 50 may be fitted into the two respective cutouts 23 of the substrate 22 in place of the positioning pins 41.

Variation 2 is not limited to the aforementioned example in which the attaching member 50 is made of a resin material. For example, the attaching member 50 may be made of a metallic material.

Variation 2 is not limited to the aforementioned example in which the clearance SP3 is defined between the screw head 51 and the first principal surface PS1 of the substrate 22. For example, the screw head 51 may be in contact with the first principal surface PS1 of the substrate 22, without the clearance SP3 therebetween.

### [Variation 3]

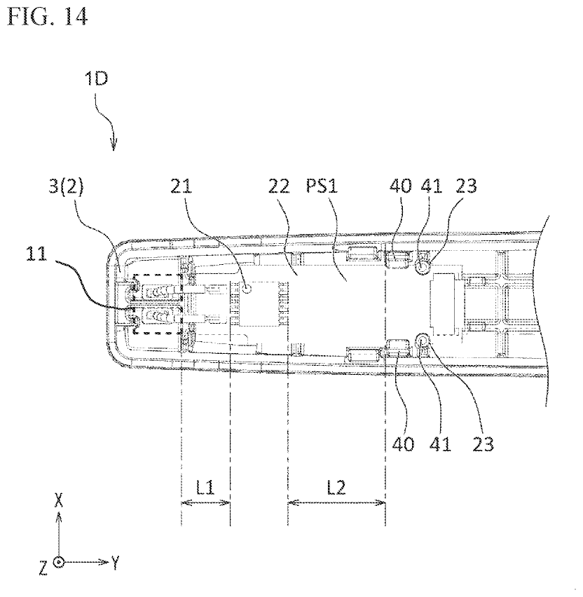

FIG. 14 schematically illustrates the configuration of a moisture measuring device 1D according to Variation 3. As illustrated in FIG. 14, the processing unit 21 is disposed closer to the biosensor 11 in the moisture measuring device 1D than in the moisture measuring device 1A according to Embodiment 1. The attaching members 40 are disposed farther from the biosensor 11 than is the processing unit 21 and face each other. The other configuration of the moisture measuring device 1D according to Variation 3 is identical to that of the moisture measuring device 1A according to Embodiment 1.

When viewed in the direction (the Z direction) perpendicular to the first principal surface PS1 of the substrate 22, the processing unit 21 of the moisture measuring device 1D is located on the side closer to the biosensor 11 with respect to the midpoint in the longitudinal direction (the Y direction) of the substrate 22. This configuration results in a shorter distance between L1 the processing unit 21 and the biosensor 11. Accordingly, noise is less likely to be introduced into the processing unit 21.

When viewed in the direction (the Z direction) perpendicular to the first principal surface PS1 of the substrate 22, the attaching members 40 are located on the opposite side from the biosensor 11 with respect to the midpoint in the longitudinal direction (the Y direction) of the substrate 22. This configuration results in an increase in the distance L2 between the processing unit 21 and the attaching members 40. Accordingly, the noise caused by capacitive coupling at the attaching members 40 is less likely to be introduced into the processing unit 21.

The distance L2 between the attaching members 40 and the processing unit 21 is greater than the distance L1 between the biosensor 11 and the processing unit 21 when viewed in the direction (the Z direction) perpendicular to the first principal surface PS1 of the substrate 22. Referring to FIG. 14, the distance L1 is the shortest distance between an end portion (a right end) of the biosensor 11 closer to the processing unit 21 and an end portion (a left end) of the processing unit 21 closer to the biosensor 11. The distance L2 is the shortest distance between an end portion (a right end) of the processing unit 21 closer to the attaching members 40 and end portions (left ends) of the attaching members 40 closer to the processing unit 21. With this configuration, noise is much less likely to be introduced into the processing unit 21. As a result, the measurement accuracy of the moisture measuring device 1D can be further improved.

### EXAMPLES

The following describes examples.

As mentioned above, creating the clearance SP1 reduces the possibility of the damage to the substrate 22 subjected to, for example, a drop impact and also reduces the possibility of capacitive coupling. Accordingly, drop tests were conducted with varying relationships between the thickness of the substrate 22 and the size of the clearance SP1 to evaluate whether the substrate 22 was damaged.

### Experiment 1

Drop tests were conducted in Experiment 1, where a housing being a cube with six sides and fitted with the substrate 22 was dropped from a height of one meter and hit a concrete ground, with the substrate 22 being screwed onto any one of the sides of the six sides of the housing. In the drop tests, the housing was dropped with each side of the housing facing down, once for each side.

Experiment 1 involved drop tests conducted with varying sizes of the clearance SP1, namely, 0 mm, 0.1 mm, 0.3 mm, and 0.5 mm, for a substrate 22 having a thickness of 1 mm to evaluate whether the substrate 22 was damaged.

Experiment 1 also involved calculation of measurement errors in moisture content measurements conducted using the moisture measuring device 1A according to Embodiment 1 with varying sizes of the clearance SP1, namely, 0 mm, 0.1 mm, 0.3 mm, and 0.5 mm, for a substrate 22 having a thickness of 1 mm.

### Experiment 2

Experiment 2 involved drop tests conducted in the same manner as in Experiment 1 with varying sizes of the clearance SP1, namely, 0 mm, 0.12 mm, 0.3 mm, and 0.6 mm, for a substrate 22 having a thickness of 1.2 mm to evaluate whether the substrate 22 was damaged.

Experiment 2 also involved calculation of measurement errors in moisture content measurements conducted using the moisture measuring device 1A according to Embodiment 1 in the same manner as in Experiment 1 with varying sizes of the clearance SP1, namely, 0 mm, 0.12 mm, 0.3 mm, and 0.6 mm, for a substrate 22 having a thickness of 1.2 mm.

The results of Experiments 1 and 2 are shown in Tables 1 and 2.

TABLE 1

| | | |
|---|---|---|
| Size of | | |
| Clearance SP1 | Drop Test | Measurement Errors |

| | | | |
|---|---|---|---|
| 0 | mm | damaged | ±0.8% |
| 0.1 | mm | undamaged | ±0.6% |
| 0.3 | mm | undamaged | ±0.4% |
| 0.5 | mm | undamaged | ±0.4% |

TABLE 2

| | | |
|---|---|---|
| Size of | | |
| Clearance SP1 | Drop Test | Measurement Errors |

| | | | |
|---|---|---|---|
| 0 | mm | damaged | ±1.0% |
| 0.12 | mm | undamaged | ±0.8% |
| 0.3 | mm | undamaged | ±0.5% |
| 0.6 | mm | undamaged | ±0.5% |

When the clearance SP1 was 0 mm, damage was found on the substrate 22 subjected to drop tests in Experiment 1 (see Table 1). When the clearance SP1 was not less than 0.1 mm and not more than 0.5 mm, no damage was visually seen on the substrate 22 subjected to drop tests.

When the clearance SP1 was 0 mm, damage was found on the substrate 22 subjected to drop tests in Experiment 2 (see Table 2). When the clearance SP1 was not less than 0.12 mm and not more than 0.6 mm, no damage was visually seen on the substrate 22 subjected to drop tests.

The results of Experiments 1 and 2 revealed that the substrate 22 was less susceptible to, for example, a drop impact when the size of the clearance SP1 was not less than 0.1 times the thickness of the substrate 22 and not more than 0.5 times the thickness of the substrate 22.

The substrate 22 may somewhat be thick to resist bending under, for example, a drop impact. Given that solder cracks can occur due to the bending of the substrate 22 with electronic components soldered thereto, increasing the thickness of the substrate 22 to reduce the possibility of, for example, solder cracks leads to an improvement in product quality.

An increase in the size of the clearance SP1 allows the dielectric to be located farther from the pattern on the substrate 22, thus suppressing parasitic capacitance. As can be seen from Tables 1 and 2, measurement errors arising from the use of the moisture measuring device 1A were smaller when the clearance SP1 was larger in size.

While the present disclosure has been thoroughly described so far in relation to the preferred embodiments with reference to the accompanying drawings, variations and modifications will be apparent to those skilled in the art. It should be understood that the variations and modifications made without departing from the scope hereinafter claimed are also embraced by the present disclosure.

The moisture measuring device according to the present disclosure is applicable to, for example, moisture measuring devices such as oral moisture checkers and skin moisture checkers.
1A, 1B, 1C, 1D moisture measuring device
2 housing
3 first housing
4 second housing
10 sensor part
12 sensor surface
20 probe part
21 processing unit
22 substrate
23 cutout
24 conductor part
25 through-hole
30 grip part
31 operation display unit
32 calculation unit
40 attaching member (engagement claw)
41 positioning pin
42 protrusion
43 support member
44 threaded hole
50 attaching member (screw)
51 screw head
52 screw main body
D1 longitudinal direction
E1 one end
E2 other end
L1, L2 distance
P1 spacing
PS1 first principal surface
PS2 second principal surface
R1, R2 overlap region
SP1 clearance
SP2 space
SP3 clearance

1. A moisture measuring device comprising:
a biosensor having a sensor surface for acquisition of biological information;
a processing unit configured to process the biological information acquired by the biosensor;
a substrate having a first principal surface on which the processing unit is mounted;
a housing accommodating the biosensor, the processing unit, and the substrate; and
one or more attaching members disposed on the housing and facing the first principal surface of the substrate to attach the substrate to the housing, wherein
a total area of one or more overlap regions in which the one or more attaching members overlap the substrate in the first principal surface of the substrate when viewed in a direction perpendicular to the first principal surface of the substrate is smaller than a sensor area of the sensor surface of the biosensor.

2. The moisture measuring device according to claim 1, wherein
the first principal surface of the substrate is provided with a conductor part including a wiring pattern, and
the total area of the one or more overlap regions is smaller than a total area of the conductor part.

3. The moisture measuring device according to claim 1, wherein the one or more attaching members comprise a resin.

4. The moisture measuring device according to claim 1, wherein the one or more attaching members include one or more screws each having a screw head.

5. The moisture measuring device according to claim 1, wherein
the one or more attaching members include one or more engagement claws, and
the one or more engagement claws each have a protrusion located in the housing, facing the first principal surface of the substrate, and protruding inward from outside the substrate.

6. The moisture measuring device according to claim 5, wherein the one or more engagement claws are located apart from the first principal surface of the substrate.

7. The moisture measuring device according to claim 6, wherein a distance between each of the one or more engagement claws and the first principal surface of the substrate is not less than 0.1 times a thickness of the substrate and not more than 0.5 times the thickness of the substrate.

8. The moisture measuring device according to claim 5, wherein
the substrate has a longitudinal direction and a lateral direction, and
the engagement claws are disposed at ends on opposite sides in the lateral direction of the substrate.

9. The moisture measuring device according to claim 8, wherein the engagement claws are arranged with a spacing provided therebetween in the longitudinal direction of the substrate.

10. The moisture measuring device according to claim 9, wherein the engagement claws include
a first engagement claw disposed between the biosensor and the processing unit in the longitudinal direction of the substrate, and
a second engagement claw disposed on an opposite side from the first engagement claw in the lateral direction of the substrate and closer to the processing unit than is the first engagement claw in the longitudinal direction of the substrate.

11. The moisture measuring device according to claim 1, wherein
the substrate has a second principal surface located opposite to the first principal surface,
the housing includes a support member by which the second principal surface is partially supported, and
a space is defined between the housing and the substrate.

12. The moisture measuring device according to claim 1, wherein the one or more attaching members are disposed farther from the biosensor than is the processing unit.

13. The moisture measuring device according to claim 12, wherein a distance between each of the one or more attaching members and the processing unit is greater than a distance between the biosensor and the processing unit.

14. The moisture measuring device according to claim 1, wherein
the biosensor is a capacitive sensor configured to measure electrostatic capacity, and
the processing unit is configured to perform conversion processing in which the electrostatic capacity measured by the capacitive sensor is converted into frequency.

15. The moisture measuring device according to claim 2, wherein the one or more attaching members comprise a resin.

16. The moisture measuring device according to claim 2, wherein the one or more attaching members include one or more screws each having a screw head.

17. The moisture measuring device according to claim 3, wherein the one or more attaching members include one or more screws each having a screw head.

18. The moisture measuring device according to claim 2, wherein
the one or more attaching members include one or more engagement claws, and
the one or more engagement claws each have a protrusion located in the housing, facing the first principal surface of the substrate, and protruding inward from outside the substrate.

19. The moisture measuring device according to claim 3, wherein
the one or more attaching members include one or more engagement claws, and
the one or more engagement claws each have a protrusion located in the housing, facing the first principal surface of the substrate, and protruding inward from outside the substrate.

20. The moisture measuring device according to claim 4, wherein
the one or more attaching members include one or more engagement claws, and
the one or more engagement claws each have a protrusion located in the housing, facing the first principal surface of the substrate, and protruding inward from outside the substrate.
